(19) United States
(12) Plant Patent Application Publication
(10) Pub. No.: US 2006/0225168 P1
(43) Pub. Date: Oct. 5, 2006

(54) Arbequina IRTA I-18 olive tree

(50) OLEA EUROPAEA; OLIVE

(76) Inventor: Joan Tous, Subirats (ES)

Correspondence Address: MARK D MILLER;KIMBLE, MACMICHAEL & UPTON, 5260 NORTH PALM AVENUE, SUITE 221, FRESNO, CA, 93704, US

(21) Appl. No.: 11/093,398

(22) Filed: Mar. 29, 2005

(51) Int. Cl. A01H 5/00

(52) U.S. Cl. PLT/158

(57) ABSTRACT

This new invention of an olive tree is characterized by its similarity to the ‘Arbequina Standard’ from which it is a clonal selection from which it is distinguished higher productivity, larger fruit size, better pulp to stone ratio, higher oil yield, and better oil quality. The plants are ideally suited for mechanical harvesting and high density planting. The fruit matures in northern California during the first week of October.

### BACKGROUND OF THE NEW VARIETY

The present invention relates to a new and distinct variety of olive plant, Olea europaea, and will hereafter be denominated varietally as ‘Arbequina IRTA I-18’ and more particularly to a olive plant that produces fruit for harvest during the first week of October in the Gridley area of the northern Sacramento Valley in California and which is further distinguished as to novelty by producing a fruit with higher productivity than ‘Arbequina Standard’ variety. The maturing date in Northern California is different than in Spain but is materially the same with better oil quality.

### ORIGIN AND ASEXUAL REPRODUCTION OF THE NEW VARIETY

The ‘Arbequina IRTA I-18’ was originally selected from 110 individual trees from olive orchards across Spain for possessing superior traits in 1986. Four years later, fifteen (15) advanced further selections were made that possessed superior production and uniformity in ripening. Two years later, these advanced selections were propagated vegetatively and placed in replicated trials. In 1997, the new variety evaluation provided the information, on its superior traits over the Arbequina Standard. The new invention was imported into Oregon, USA where it was grown and then vegetatively propagated and shipped to Gridley, Calif. Over the past three years propagational trees were provided as rooted cuttings. The fruit produced by these vegetatively propagated trees are in all respects identical to the parent.

### SUMMARY OF THE NEW VARIETY

This new variety of olive tree is characterized by having the general characteristics of the ‘Arbequina Standard’ variety with several differences including: semi-erect tree (rather than open), fruit has smaller pits, higher oil content and larger fruit. Due to the semi-erect structure and size of this olive tree, it is well suited for mechanical harvesting and high density planting (i.e., 670 plants/acre).

### BRIEF DESCRIPTION OF THE DRAWINGS

FIG. 1 is a color photograph of the olive tree at third leaf (three years old).

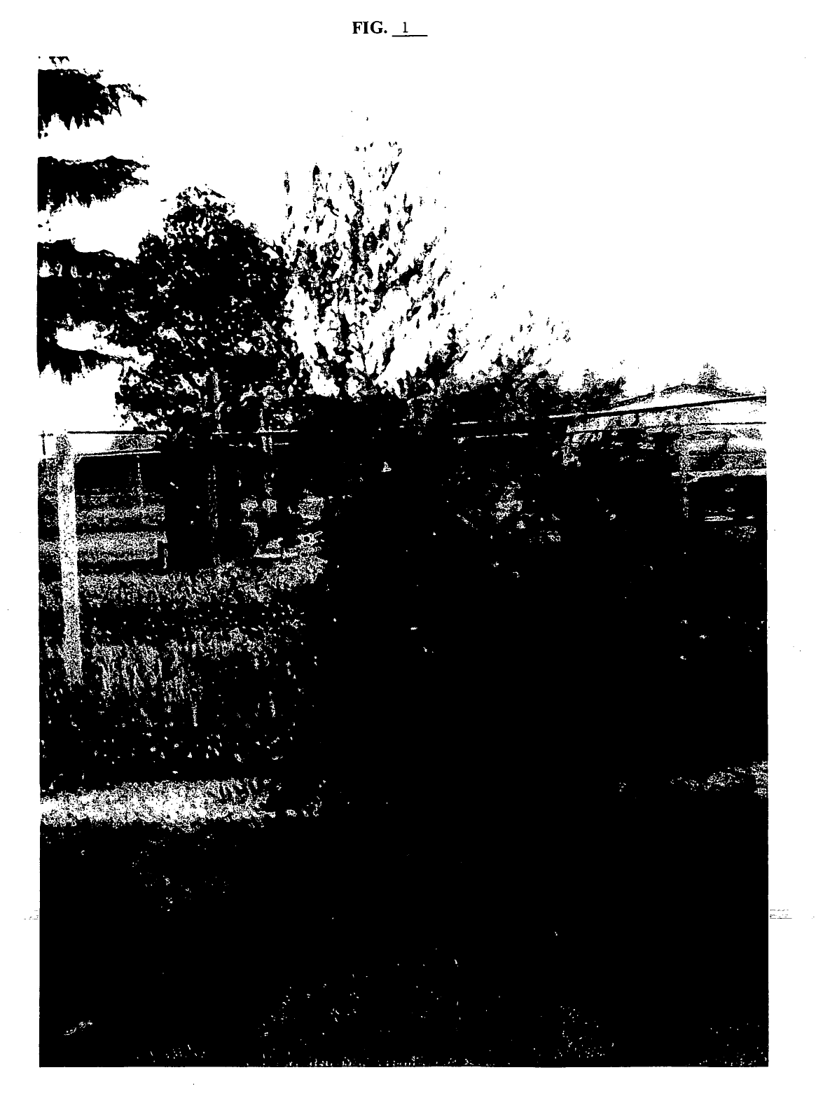

FIG. 2 is a color photograph of the new invention showing it in the third leaf displaying it semi-erect in appearance.

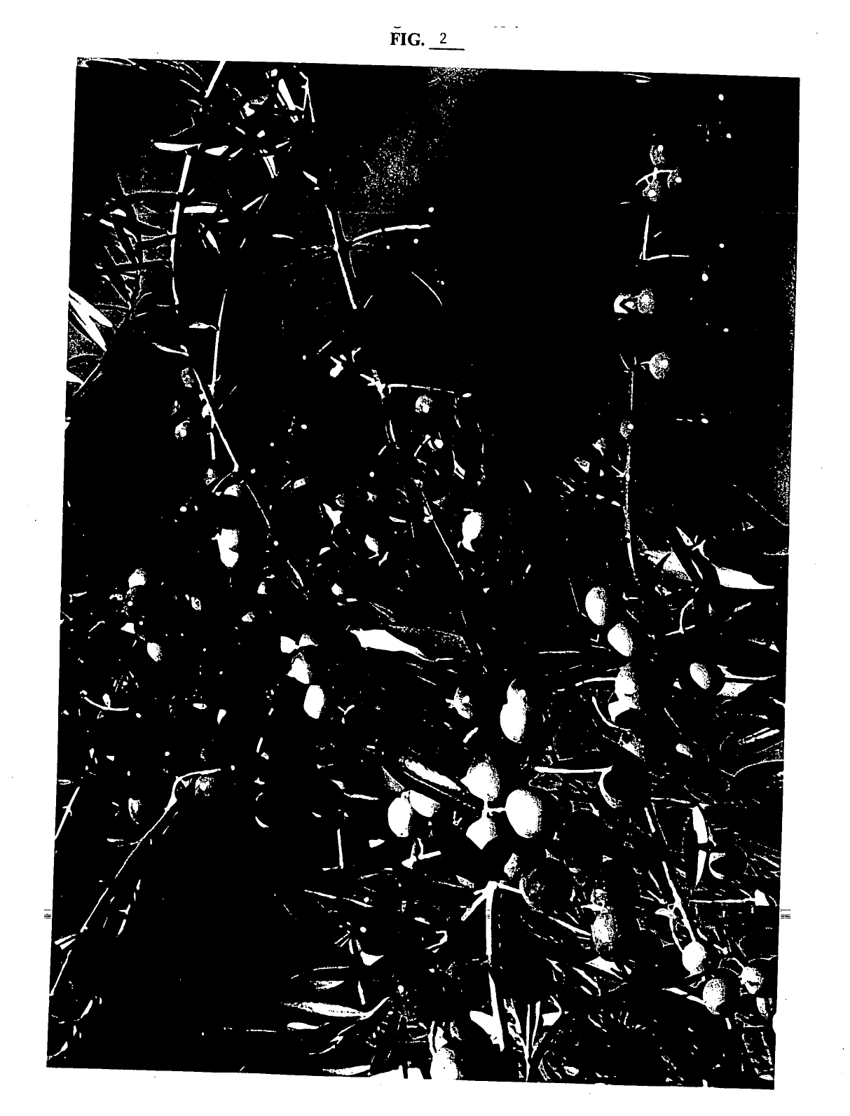

FIG. 3 is a color photograph showing a display of the fruit clusters, twigs and branches.

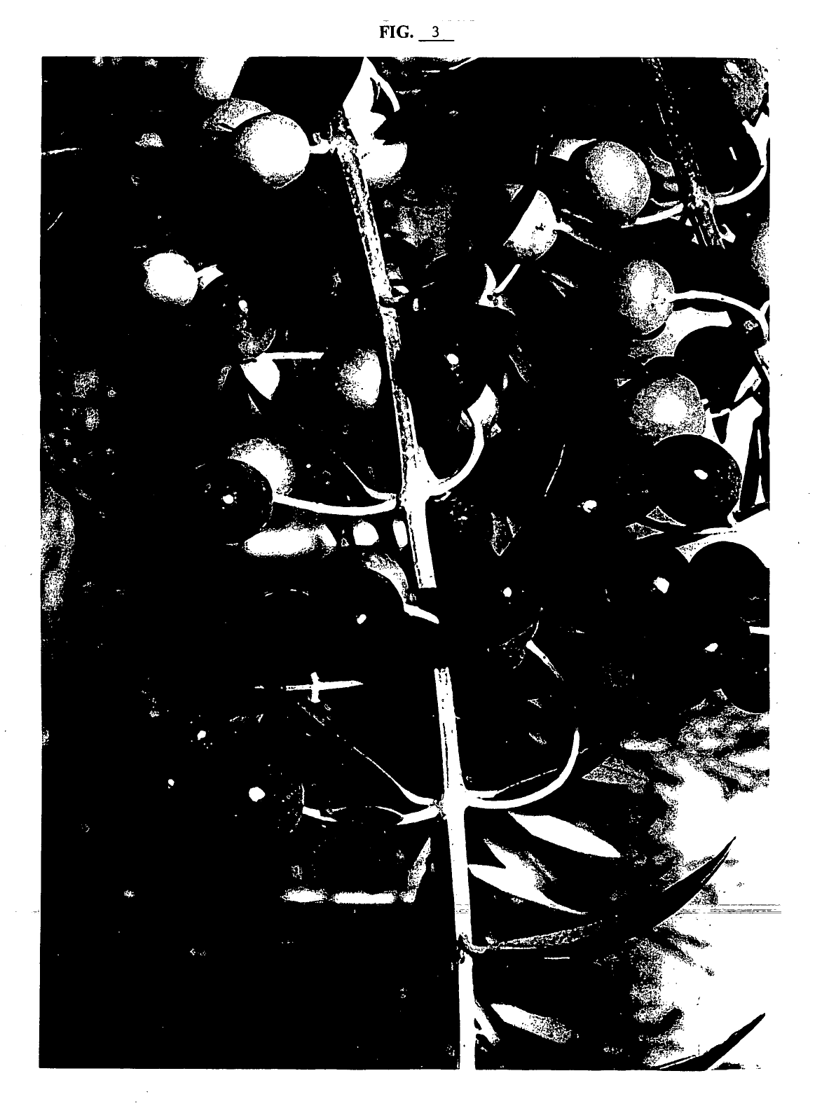

FIG. 4 is a color photograph showing two whole fruits displaying the shape of the fruit (top), a fruit cut in half with seed in place (lower left), another fruit cut in half displaying the flesh and pit well (lower right), and a seed in dry state (lower center).

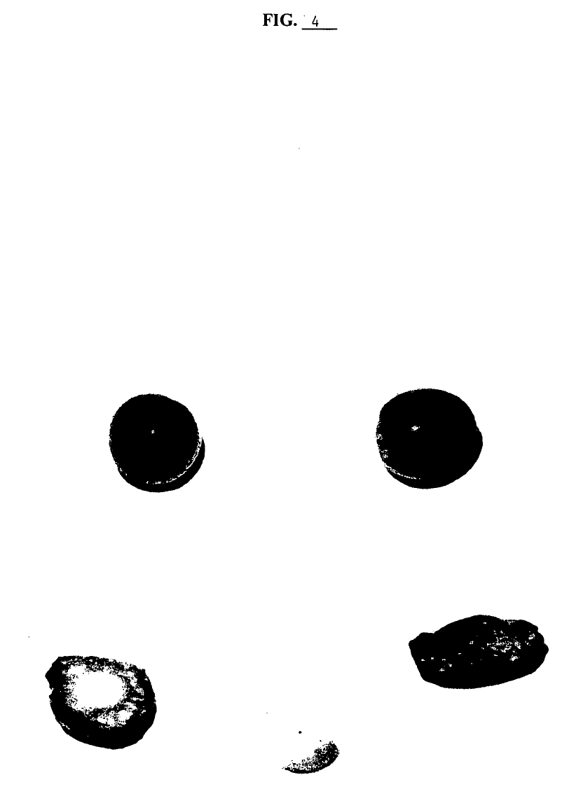

### DETAILED DESCRIPTION

Referring more specifically to the horticultural details of the new and distinct variety of olive trees, the following descriptions have been observed under the ecological details prevailing at the site of origin which is located near Gridley, Calif. in the Sacramento Valley of Northern California. All major color designations are by reference to the Dictionary of color by Maerz & Paul, First Edition, 1930. Common colors are also employed.
Tree:
   Size.—small, semi-erect. Height: 9-10 feet (274-305 cm.) Width: 2-3½ feet (61-107 cm.).
   Vigor.—medium, good.
   Chilling requirements.—normal for olives.
   Shape.—upright, semi-erect.
   Productivity.—excellent.
   Regularity of bearing.—regular.
Trunk:
   Size.—circumference of 6 inches, 11 inches above ground.
   Surface texture.—smooth.
   Color.—Plumbago Grey (44-B-4).
   Lenticels.—Number: scattered, irregularly spaced Size: very small.
Branches:
   Size.—small, circumference of 2-3 inches (5-8 cm), 15 inches (38 cm) above crotch.
   Surface texture.—Mature: smooth Immature: smooth.
   Color of branches one year or older.—Grey Drab, Quaker Grey (1-B-1).
   Color of branches less than one year old (immature).—Light Grey (36-A-1).
   Lenticels.—Numbers: few and scattered Size: very small.
Leaves:
   Configuration.—Leaves on opposite side of the stem.
   Size.—small Length: 45-49 mm (1.76-1.91 in.) Width: 10-12 mm (0.39-0.47 inch).
   Shape.—lancelate.
   Color.—Upwardly disposed surface: Fir (glossy) (24-E-7) Downwardly disposed surface: Cosse Green (19-L-5).
   Marginal form.—entire, complete.
   Leaf vein.—Color: Chrysolite Green (19-K-3) Thickness: less than 1 mm (0.039 in.).
   Glandular characteristics.—none.
   Petiole.—Size: medium Length: 4-6 mm (0.16-0.23 in.) Thickness: 1.5 mm (0.06 in.) in diameter Color Code— Palm Leaf (14-L-1).
   Stem glands.—none.
   Stipules.—none.
   Flower buds.—small Surface texture: slightly pubescent.
Flowers:
   Date of first bloom.—mid May to end of May.
   Size.—small, 40-60 mm (0.16 in.-0.24 in.) — numbers do not add up Petals: Color: White.
Fruit:
   Date of maturity.—first week of October, in clusters of 2-3.
   Size.—small, 2.85 grams Diameter: 14-15 mm (0.55-0.59 in.) Length: 16-18 mm (0.62-0.70 in.).
   Form.—Uniformity: good Symmetrical or Asymmetric: slightly asymmetrical Suture: none Stem cavity: Width: 6 mm (0.23 in.) Depth: 3 mm (0.12 in.) Length: 10 mm (0.39 in.)
   Stem.—Short Caliper: 1.5 mm (0.06 in.)
   Apex.—rounded.
   Pistal point.—rounded.
   Skin.—Thickness: thin Texture: smooth Ground color: From Byzantium (44-K-7) to Kurdistan (48-A-10) Flesh color: Tinsel Deepstone (13-L-7) Color of surface of pit cavity: Sudan (13-E-4) Color of pit well: Sudan (13-E-4).
   Juice production.—Very Good.
   Flavor.—very good.
   Aroma.—none.
   Texture.—smooth, firm.
   Fibers.—few.
   Ripening.—even.
   Eating quality.—used for oil production and in some cases for canning.
Seed:
   Free or cling.—Cling.
   Fibers.—few.
   Size.—Length: 12 mm (0.47 inch) Width: 7 mm (0.28 inch) Thickness: 5 mm (0.20 inch).
   Form.—Elliptical.
   Apex shape.—rounded with a point Color (dry): Peruvian Brown (13-L-11).
   Base.—rounded.
   Sides.—slightly asymmetrical.
   Ridges.—not uniform.
   Tendency to split.—none known.
   Use.—olive oil production and in some cases for canning.
   Keeping and shipping quality.—good.
   Harvesting.—first week of October.

### Chemical Analysis (Characteristics)

### ‘Arbequina IRTA I-18’

TABLE 1
Virgin Oil Fatty Acid Percentage

| | Percent of Total Oil |
|---|---|
| Type of Fatty Acid | Content |
| C16:0 Palmitic Acid | 16.0 |
| C16:1 Palmitoleic Acid | 1.4 |
| C18:0 Esteric Acid | 1.5 |
| C18:1 Oleic Acid | 71.1 |
| C18:2 Linoleic Acid | 7.66 |
| C18:3 Linoleic Acid | 0.71 |

M/P ratio (monosaturated fats/polysaturated fats).—6.2 .
Polyphenols (ppm catteic acid).—0.25.
K225 (bitterness).—0.0125.

What is claimed is:

1. A new distinct olive plant as described and illustrated that is characterized by having the general characteristics of the ‘Arbequina Standard’ variety but is distinguished therefrom in that it has higher productivity, larger fruit size, better pulp to stone ratio, and higher oil yield, the plants being ideally suited for mechanical harvesting and high density planting, with the fruit maturing in northern California in about the first week of October.
